(19) United States
(12) Patent Application Publication
(10) Pub. No.: US 2023/0078757 A1
(43) Pub. Date: Mar. 16, 2023

(54) HYDRAULIC IMPLANT CRIMPING SYSTEMS AND METHODS

(71) Applicant: Edwards Lifesciences Corporation, Irvine, CA (US)

(72) Inventor: Raffi Sempad Pinedjian, Fountain Valley, CA (US)

(21) Appl. No.: 17/935,860

(22) Filed: Sep. 27, 2022

Related U.S. Application Data
(63) Continuation of application No. PCT/US2021/023716, filed on Mar. 23, 2021.
(60) Provisional application No. 63/004,240, filed on Apr. 2, 2020.

(51) Int. Cl. A61F 2/24

(52) U.S. Cl. CPC A61F 2/2415; A61F 2/2427

(57) ABSTRACT

Apparatuses, systems, and methods for crimping implants. The apparatuses in certain embodiments may comprise crimping devices for compressing an implant prior to deployment to a portion of a subject. The crimping devices may apply a hydraulic force radially upon the implant within an implant receiving region to compress the implant.

This is a continuation of PCT patent application no. PCT/US2021/023716, filed on Mar. 23, 2021, which application claims priority to U.S. provisional patent application No. 63/004,240, filed on Apr. 2, 2020, each of these applications being incorporated by reference herein in its entirety.

Certain embodiments disclosed herein relate to apparatuses, systems, and methods for crimping implants. The apparatuses in certain embodiments may comprise crimping devices for compressing an implant prior to deployment to a subject.

### BACKGROUND

Human heart valves, which include the aortic, pulmonary, mitral and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include a tissue-based valve body that is connected to an expandable frame that is then delivered to the native valve's annulus.

Development of prostheses including but not limited to replacement heart valves that can be compacted for delivery and then controllably expanded for controlled placement has proven to be particularly challenging. A delivery apparatus may be provided to deploy such an implant to the desired location in the human body. The implant may be in a compressed state when coupled to the delivery apparatus, and thus must be compressed for delivery to the desired location of implantation within the patient's body. Methods exist to crimp such implants prior to delivery, however, it may be desirable to provide improved apparatuses, systems, and methods.

### SUMMARY

Embodiments of the present disclosure are directed to crimping devices for an implant. In certain embodiments, a crimping device may include an implant receiving region. The crimping device may include one or more bodies positioned about the implant receiving region and configured to apply a hydraulic force radially upon the implant within the implant receiving region to compress the implant.

Embodiments of the present disclosure are directed to systems. In certain embodiments, a system may include a delivery apparatus configured to deliver an implant to a location within a subject and including an elongate shaft having an implant retention area. The system may include an implant configured to be compressed and expanded, and configured to be retained at the implant retention area and deployed to the location within the subject. The system may include a crimping device having an implant receiving region and one or more bodies configured to apply a hydraulic force radially upon the implant within the implant receiving region to compress the implant.

Embodiments of the present disclosure are directed to methods. In certain embodiments, a method may include positioning an implant within an implant receiving region of a crimping device, the crimping device including one or more bodies configured to apply a hydraulic force to the implant. The method may include applying the hydraulic force radially upon the implant with the one or more bodies to compress the implant within the implant receiving region.

### BRIEF DESCRIPTION OF THE DRAWINGS

Features and advantages of the systems, apparatuses, and methods as disclosed herein will become appreciated as the same become better understood with reference to the specification, claims, and appended drawings wherein:

FIG. 1 illustrates a perspective view of a crimping device according to an embodiment of the present disclosure.

FIG. 2 illustrates a cross sectional view of the crimping device shown in FIG. 1.

FIG. 3 illustrates a side cross sectional view of the crimping device shown in FIG. 1.

FIG. 4 illustrates the side cross sectional view of the crimping device shown in FIG. 3 with an implant positioned within an implant receiving region.

FIG. 5 illustrates the side cross sectional view of the crimping device shown in FIG. 4 with the implant crimped.

FIG. 6 illustrates a perspective view of a crimping device according to an embodiment of the present disclosure.

FIG. 7 illustrates a cross sectional view of the crimping device shown in FIG. 6.

FIG. 8 illustrates a cross sectional view of the crimping device shown in FIG. 6 moved to an open state.

FIG. 9 illustrates a cross sectional view of a crimping device according to an embodiment of the present disclosure.

FIG. 10 illustrates a perspective view of a crimping device according to an embodiment of the present disclosure.

FIG. 11 illustrates a cross sectional view of the crimping device shown in FIG. 10.

FIG. 12 illustrates a schematic view of a crimping device according to an embodiment of the present disclosure.

FIG. 13 illustrates a side view of an implant according to an embodiment of the present disclosure.

FIG. 14 illustrates a perspective view of an implant according to an embodiment of the present disclosure.

FIG. 15 illustrates a perspective view of an implant according to an embodiment of the present disclosure.

FIG. 16 illustrates a perspective view of the implant shown in FIG. 15 with an outer covering removed according to an embodiment of the present disclosure.

FIG. 17 illustrates a perspective view of a frame of the implant shown in FIG. 15 in a compressed state according to an embodiment of the present disclosure.

FIG. 18 illustrates a side view of a delivery apparatus according to an embodiment of the present disclosure.

FIG. 19 illustrates a schematic view of a delivery apparatus approaching a heart valve according to an embodiment of the present disclosure.

FIG. 20 illustrates a schematic view of an implant deployed to a heart valve according to an embodiment of the present disclosure.

FIG. 21 illustrates a side partial cross sectional view of a hydraulic actuator according to an embodiment of the present disclosure.

FIG. 22 illustrates a side partial cross sectional view of the hydraulic actuator shown in FIG. 21 according to an embodiment of the present disclosure.

### DETAILED DESCRIPTION

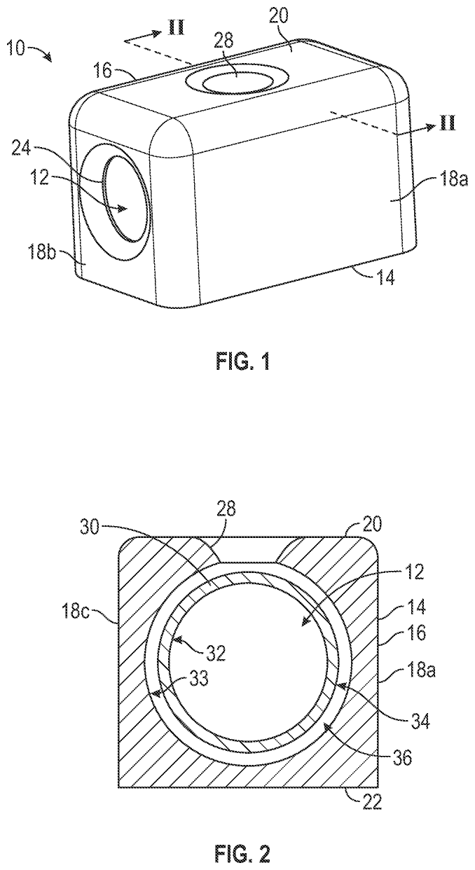

FIG. 1 illustrates a perspective view of a crimping device 10 for an implant. The crimping device 10 may include an implant receiving region 12 and may include a body 14 that is positioned about the implant receiving region 12 and is configured to apply a hydraulic force radially upon an implant within the implant receiving region 12 to compress the implant.

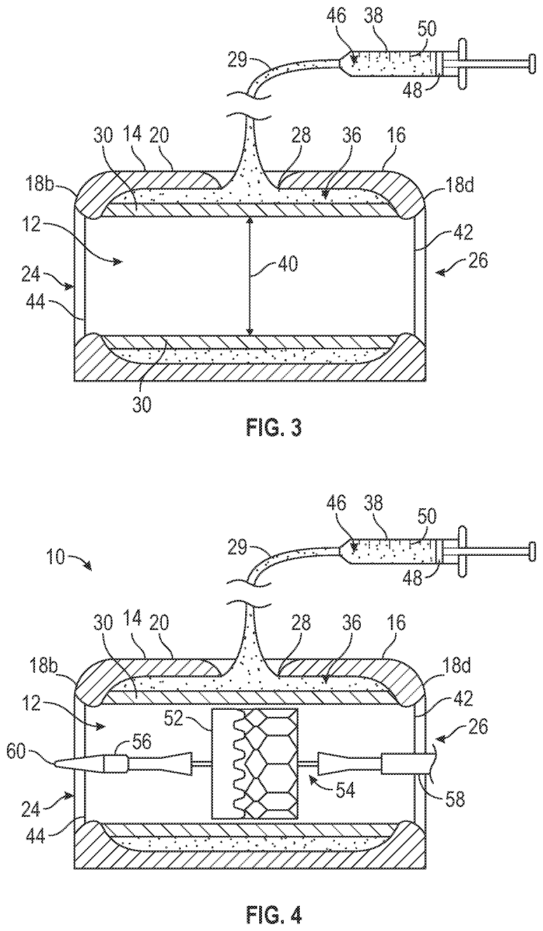

The body 14 may include a housing 16 that includes a plurality of side walls 18a-d (side wall 18c is marked in FIG. 2, and side wall 18d is marked in FIG. 3) that may form the outer side surfaces of the crimping device 10. The housing 16 may include a top wall 20 forming an upper outer surface of the housing 16, and may include a bottom wall 22 (marked in FIG. 2) that may form an outer lower surface of the housing 16. The housing 16 may have a rectangular shape as shown in FIG. 1, or may have another shape as desired (e.g., square, cylindrical, trapezoidal, among others). The housing 16 may be constructed of rigid materials if desired.

The housing 16 may include an opening 24 at an end of the housing 16, which may be at the side wall 18b. The housing 16 may include an opening 26 (marked in FIG. 3) at an opposite end of the housing 16, which may be at the side wall 18d (marked in FIG. 3). The openings 24, 26 may be configured for an implant, and at least a portion of a delivery apparatus, to be inserted through to be positioned within the implant receiving region 12. The openings 24, 26 may be positioned coaxial with each other, at opposite ends of the implant receiving region 12.

The housing 16 may include a port 28 that is configured for fluid transfer to or from the body 14. The port 28 may be configured to couple with one or more fluid conduits 29 (as marked in FIG. 3) that may allow for fluid transfer to or from the body 14 via the port 28. The port 28 may be positioned on the outer upper surface of the housing 16 as shown in FIG. 1, or may be positioned in other locations as desired.

A cross sectional view along line II-II in FIG. 1 is shown in FIG. 2. The body 14 may include a movable wall 30 positioned within the housing 16 and surrounding the implant receiving region 12. The movable wall 30 may form a sleeve around the implant receiving region 12. The movable wall 30 may comprise a flexible material that is configured to move to apply a compressive force upon an implant positioned within the implant receiving region 12. The moveable wall 30 may include an inner surface 32 that faces towards the implant receiving region 12 and is a compressive surface that is configured to apply a compressive force to the implant within the implant receiving region. In embodiments, the inner surface 32 may be made of the same material as the remainder of the movable wall 30 or may include a layer of material. For example, a lubricous layer of material such as polytetrafluoroethylene (PTFE) or Qualcrimp®, or another form of lubricous material may be utilized as the inner surface 32 as desired. The movable wall 30 may include an outer surface 34 that faces opposite the inner surface 32 and away from the implant receiving region 12. The outer surface 34 may face towards the inner surface 33 of the housing 16 and towards a fluid chamber 36 positioned around the movable wall 30.

The fluid chamber 36 may be positioned between the movable wall 30 and the housing 16 and may be configured to retain fluid that presses against the outer surface 34 of the movable wall 30. The movable wall 30 accordingly may be positioned between the implant receiving region 12 and the fluid chamber 36. The fluid chamber 36 may surround the movable wall 30 and the implant receiving region 12. The fluid chamber 36 may extend along the length of the movable wall 30 and the housing 16. The fluid chamber 36 may accordingly form a cylindrical ring extending along the length of the body 14 and surrounding the movable wall 30 and the implant receiving region 12.

The fluid chamber 36 may be in fluid communication with the port 28 and accordingly may be filled with fluid or may have fluid withdrawn from the fluid chamber 36 via the port 28 (and through the fluid conduit 29 as marked in FIG. 3). The fluid chamber 36 may be filled with fluid to cause the pressure of the fluid to press against the outer surface 34 of the movable wall 30 and move the wall 30 towards the center of the implant receiving region 12 and thus compressing an implant positioned within the implant receiving region 12. The body 14 accordingly may be an expandable body that expands in size due to the amount of fluid within the fluid chamber 36. The compressive force upon the implant may be a hydraulic compressive force that is applied radially upon the implant. For example, an implant positioned within the implant receiving region 12 will experience a force applied radially due to the position of the movable wall 30. The body 14 is configured to be filled with fluid to apply the hydraulic force radially upon the implant. The body 14 is configured to be inflated with fluid to reduce the size of the implant receiving region 12 and to apply the hydraulic force radially upon the implant within the implant receiving region 12.

The fluid chamber 36 may be configured for fluid to be withdrawn from the fluid chamber 36 and accordingly from the body 14. Such an operation may reduce the size of the body 14 and increase the size of the implant receiving region 12. Fluid may be withdrawn from the port 28 via a fluid conduit 29 (as marked in FIG. 3) or via another method to withdraw the fluid from the body 14. The size of the implant receiving region 12 may be increased to remove the crimped implant from the implant receiving region 12 and to allow for reuse of the crimping device 10 for crimping another implant if desired.

FIG. 3 illustrates a side cross sectional view of the crimping device 10 shown in FIG. 1, including the fluid conduit 29 coupled to the port 28 and coupled to a hydraulic actuator 38. The movable wall 30 is shown to extend longitudinally along the length of the housing 16. The movable wall 30 has an inner diameter 40 that comprises the diameter of the implant receiving region 12. The diameter 40 is the inner diameter of the body 14. The movable wall 30 forms a tube having one end coupled to an inner surface of the housing 16 and another opposite end coupled to an inner surface of the housing 16. The movable wall 30 extends longitudinally along the length of the implant receiving region 12 and around a central axis of the implant receiving region 12. The fluid chamber 36 is further shown to extend longitudinally along the length of the housing 16 between the movable wall 30 and the housing 16, and extends longitudinally along the length of the implant receiving region 12 and the movable wall 30 and around a central axis of the implant receiving region 12 and the movable wall 30. The fluid chamber 36 is shown to be filled with fluid.

The implant receiving region 12 is shown to comprise a cylindrical area within the body 14 for receiving an implant. The implant receiving region 12 has a cylindrical shape, and the movable wall 30 forms a cylindrical shape and extends around the implant receiving region 12. The implant receiving region 12 includes an opening 42 at one end of the implant receiving region 12 and an opening 44 at another opposite end of the implant receiving region 12. The openings 42, 44 may be configured for an implant and at least a portion of a delivery apparatus to be inserted through to be positioned within the implant receiving region 12. The implant receiving region 12 may be sized to receive an implant in an uncompressed or expanded state, to be compressed to a compressed or unexpanded state.

The fluid conduit 29 is coupled to the body 14 and is configured to transfer fluid to or from the body 14. One end of the fluid conduit 29 may be coupled to the port 28 and another end may be coupled to the hydraulic actuator 38. The fluid conduit 29 may comprise tubing that is configured to transfer fluid or may have another form as desired.

The hydraulic actuator 38 may be configured to transfer fluid to or from the body 14. The hydraulic actuator 38 may include a fluid chamber 46 and a piston 48 that is configured to move within the fluid chamber 46. The movement of the piston 48 may cause the fluid to be moved into and out of the fluid chamber 46 and accordingly into or out of the fluid chamber 36 of the body 14. The hydraulic actuator 38 may be manually operated or may be automatically operated. The hydraulic actuator 38 may comprise a syringe. As such, a user may press the piston 48 (in the form of a syringe plunger) to cause the fluid chamber 36 of the body 14 to fill with fluid, and may withdraw the piston 48 to remove fluid from the fluid chamber 36 of the body 14. The fluid chamber 46 of the hydraulic actuator 38 may include indicators 50 in the form of gradations on the fluid chamber 46 for a user to determine how much fluid is within the fluid chamber 46. Other forms of hydraulic actuators may be utilized in other embodiments, for example, a variety of forms of pumps, squeeze bladders, siphons, gravity fed devices, or other forms of hydraulic actuators may be utilized.

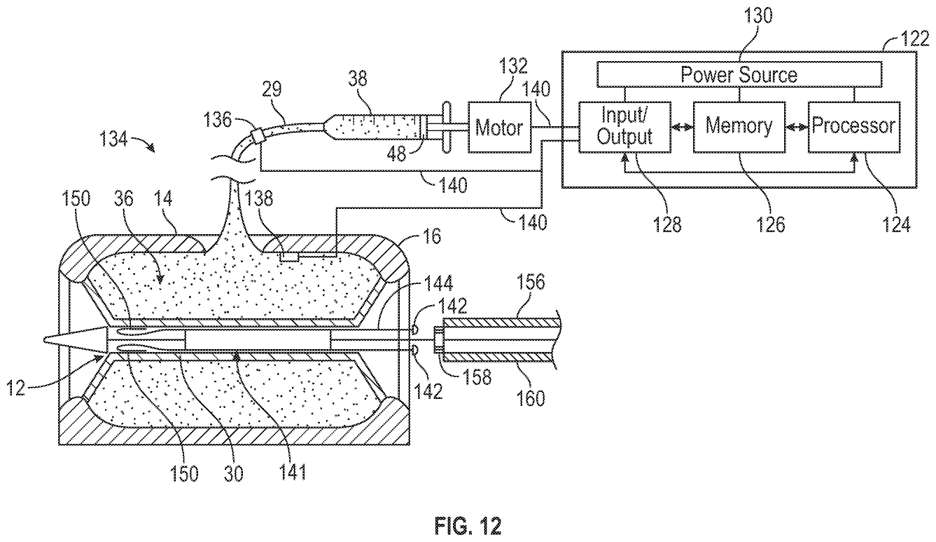

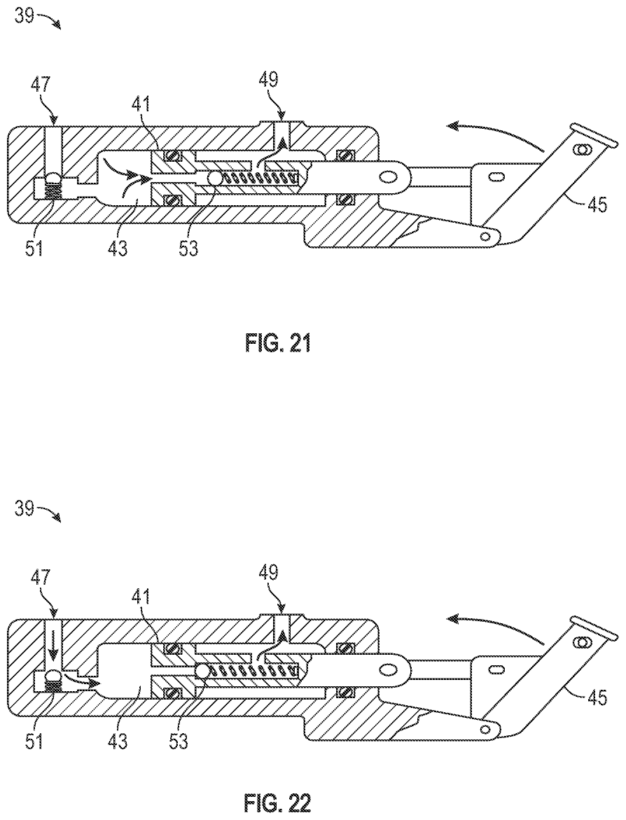

FIGS. 21 and 22 illustrate an embodiment of a hydraulic actuator 39 that may include a separate reservoir as a pressure source. Such a hydraulic actuator 39 may include a piston 41 that moves within a fluid chamber 43. A handle 45 may be operated to move the piston 41 within the fluid chamber 43. An inlet port 47 may be provided to draw fluid from a separate reservoir and outlet port 49 may be provided to pass fluid through a fluid conduit and to the body of the crimping device. Check valves 51, 53 may be provided to control flow of fluid through the hydraulic actuator 39. FIG. 21 illustrates the hydraulic actuator 39 being operated to pass fluid from the fluid chamber 43 through the outlet port 49, with check valve 51 closed and check valve 53 open. FIG. 22 illustrates the hydraulic actuator 39 being operated to pass fluid into the fluid chamber 43 from the inlet port 47 and also pass fluid out of the fluid chamber 43 through the outlet port 49. Check valve 51 is open and check valve 53 is closed. The hydraulic actuator 39 may accordingly comprise a dual action pump. The hydraulic actuator 39 may be manually operated or may be configured to be operated with a motor, for example a motor as shown in FIG. 12. The amount of fluid passed into the chamber 36 may be determined by the number of strokes of the piston 41 within the fluid chamber 43 in certain embodiments. An amount of fluid to be passed into the chamber 36 may be determined and a corresponding number of strokes of the piston 41 may be determined to correspond to that amount of fluid. The hydraulic actuator 39 may be utilized with any embodiment of crimping device disclosed herein.

Referring back to FIG. 3, the user may determine how much fluid to pass into the fluid chamber 36 and how much force to apply to the piston 48 based on the properties of the implant to be crimped. For example, for a larger diameter implant, the user may determine that less fluid is required to be passed from the fluid chamber 46. For a smaller diameter implant, the user may determine that more fluid is required to be passed from the fluid chamber 46. Further, if the implant is to be compressed to fit within a certain size of capsule of a delivery apparatus then the user may determine an appropriate amount of fluid for that size of compression. For a more fragile implant, the user may be determine that less force should be applied to the piston 48, whereas for a stiffer implant the user may determine that a greater force should be applied to the piston 48. The user may accordingly select the amount of fluid to pass into the fluid chamber 36 and how much force to apply to the piston 48 based on the properties of the implant. In embodiments, the type of hydraulic actuator 38 may be selected based on the properties of the implant to be crimped. For example, for a certain crimping diameter to be provided, a hydraulic actuator 38 may be selected having a fluid chamber 46 having a volume that matches an amount of fluid to be provided to the fluid chamber 36. As such, a user would be able to select the appropriate hydraulic actuator 38 and fully empty the fluid chamber 46 and be assured that the implant would be compressed to the desired diameter. A user may select a volume of a fluid chamber of a hydraulic actuator 38 and accordingly select an amount of fluid to fill the hydraulic actuator based on a size of compression of the implant. Further, other dimensions of the hydraulic actuator 38 may be selected to provide a desired force upon the implant.

In embodiments, the hydraulic actuator may be an auto-syringe. As such, the amount of force applied by the hydraulic actuator and the amount of fluid passed into the fluid chamber 36 may be automatically controlled by the auto-syringe. The auto-syringe may be selected based on the properties of the implant to be crimped, in a similar manner as discussed herein. In embodiments, a controller may operate the hydraulic actuator, as discussed in regard to FIG. 12.

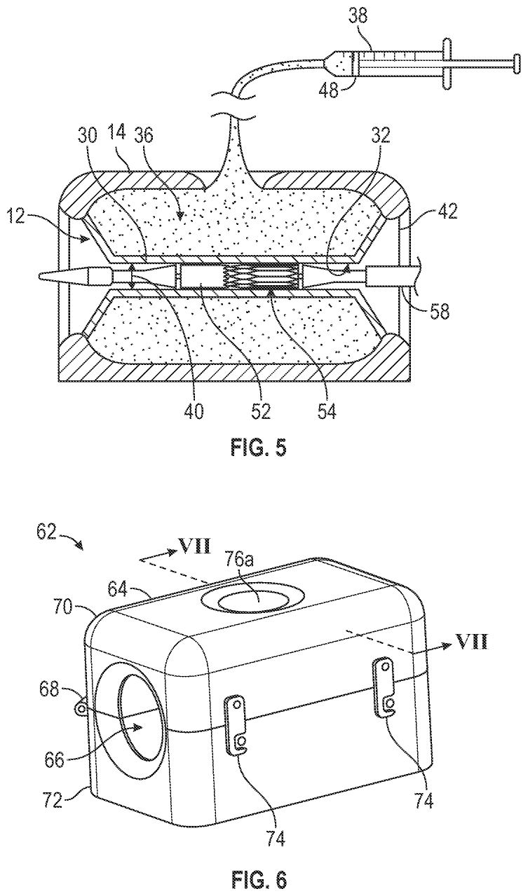

FIGS. 4 and 5 illustrate a method of operation of the crimping device 10. As shown in FIG. 4, with the movable wall 30 having a large inner diameter 40, an implant 52 may be positioned within the implant receiving region 12 of the crimping device 10. The implant 52 may comprise a compressible implant that may be configured to compress from an expanded state or deployed state to a compressed state or undeployed state. The implant 52 may be configured to be compressed and expanded, and retained at an implant retention area of a delivery apparatus, and deployed to a location within a subject. The implant 52 may comprise a prosthesis, such as a prosthetic replacement valve, including a prosthetic replacement heart valve. The prosthetic replacement heart valve may be a replacement aortic, mitral, tricuspid, or pulmonary valve. In other embodiments, other forms of prosthetic replacement valves may be utilized and other forms of implants may be utilized such as stents or other forms of medical implants. The implant 52 as shown in FIG. 4 may be a balloon expandable implant, although other forms of implants 52 may be utilized including self-expanding implants or mechanically expanded implants. The implant 52 may be inserted into the implant receiving region 12 in the expanded state or deployed state, as shown in FIG. 4. The implant 52 may be positioned within the implant receiving region 12.

The implant 52 may be positioned within an implant retention area 54 of an elongate shaft 56 of a delivery apparatus 58. The delivery apparatus 58 may be configured to deliver an implant to a location within a subject, such as a native heart valve. The elongate shaft 56 may comprise an elongate body that extends the implant to the location within the subject and may include features such as deflection of the elongate shaft 56 and a mechanism for deployment of the implant 52.

The implant receiving region 12 is configured to receive the elongate shaft 56 of the delivery apparatus 58. The elongate shaft 56 may be passed through the opening 42 as well as the opening 44 to be positioned within the implant receiving region 12. For example, the nose cone or tip 60 of the elongate shaft 56 may pass through the opening 44 with a proximal portion of the elongate shaft 56 extending through the opening 42.

A user may select an amount of fluid to fill the hydraulic actuator 38 based on a size of compression of the implant 52. For example, for a greater amount of compression a larger amount of fluid may be utilized in the hydraulic actuator 38 and for a lesser amount of compression a lesser amount of fluid may be utilized in the hydraulic actuator 38. The user may further determine how much fluid to pass into the fluid chamber 36 and how much force to apply to the piston 48 based on the properties of the implant to be crimped. A user may further select the type of hydraulic actuator 38 based on the properties of the implant to be crimped.

The hydraulic actuator 38 may then be utilized (either manually or automatically) to fill the body 14 with fluid to apply the hydraulic force radially upon the implant 52 with the body 14 to compress the implant 52 within the implant receiving region 12. The fluid chamber 36 may be filled with the fluid that is present in the fluid conduit 29 and the fluid chamber 46 to move the movable wall 30 towards the center of the implant receiving region 12. As such, a hydraulic force is radially applied upon the implant 52 with the body 14 to compress the implant 52 within the implant receiving region 12.

FIG. 5 illustrates the body 14 having compressed the implant 52 within the implant receiving region 12. The fluid chamber 36 has been filled with fluid to apply the hydraulic force radially upon the implant. The diameter 40 of the movable wall 30 and the implant receiving region 12 has decreased from the size shown in FIG. 4. The size of the implant receiving region 12 has been reduced. The movable wall 30 has moved toward the implant 52 to apply the compressive hydraulic force to the implant 52. The movable wall 30 may flex to move towards the implant 52 and may be shaped such that the inner surface 32 of the movable wall 30 forms a uniform contact surface against the implant 52. As such, as shown in FIG. 5, a uniform diameter of the movable wall 30 may be provided to distribute a uniform force against the implant 52. Further, the movable wall 30 provides a radial force symmetrically to the implant due to the movable wall 30 surrounding the outer surface of the implant 52. The movable wall 30 may be made of an elastomer or other form of flexible material that may be resilient and able to move to apply the hydraulic force to the implant 52. The implant 52 may be crimped to the implant retention area of the elongate shaft of the delivery apparatus, and may be withdrawn from the implant receiving region of the crimping device with the implant crimped to the implant retention area of the elongate shaft.

In certain embodiments, the diameter of the movable wall 30 when the fluid chamber 36 is filled with fluid may be set. For example, the movable wall 30 may be non-compliant and when the fluid chamber 36 is filled with fluid the movable wall 30 may move to a set diameter and expand no further. In such an embodiment, the non-compliance of the movable wall 30 may prevent the implant 52 from being over-compressed. The diameter of the movable wall 30 accordingly may be tailored for the particular type of implant 52 to be compressed and the particular desired diameter of compression of the implant 52. A different diameter of movable wall 30 may be utilized for a different diameter of implant 52. In other embodiments, the diameter of the movable wall 30 may be controlled by controlling the amount of fluid passed from the hydraulic actuator 38, 39.

The implant 52 is crimped upon the implant retention area 54 of the delivery apparatus 58, and may be crimped over a balloon or other inflatable body that may be utilized to expand the implant 52 at a desired time. The body 14 is configured to compress the implant 52 to the elongate shaft 56. The implant 52 may remain uncovered by another portion of the delivery apparatus 58. As such, the crimped implant 52 may be slid out of the implant receiving region 12 proximally through the opening 42 in an opposite direction that the implant 52 and delivery apparatus 58 were inserted into the implant receiving region 12. The crimped implant 52 may be slid out of the implant receiving region 12 along with the delivery apparatus 58. The crimped implant 52 may thus be in position for deployment to a subject. A lubricous layer upon the inner surface 32 of the movable wall 30 may enhance the ability of the crimped implant 52 to be slid out of the implant receiving region 12 (or slid into the implant receiving region 12 as well). In embodiments, the fluid in the fluid chamber 36 may be withdrawn from the fluid chamber 36 and accordingly from the body 14 to relieve the hydraulic force upon the implant 52 and then the implant 52 may be removed from the implant receiving region 12.

The use of a hydraulic force may provide a greater consistency for the compression of the implant, including the resulting size of the compressed implant and the force applied to the implant during compression.

Any of the crimping devices disclosed herein may be configured to be for single use application and thus may be configured to be disposable after a single use. In embodiments, portions of the crimping device may be for single use and other portions may be for multiple uses. For example, in an embodiment in which an auto-syringe is utilized, the auto-syringe may be utilized multiple times (and sterilized between uses) and the remainder of the crimping device may be disposable. In embodiments, the entirety of the crimping device may be configured for multiple uses.

FIGS. 6-8 illustrate an embodiment of a crimping device 62 in which a body 64 is configured to move from an open state in which the implant receiving region 66 is configured to receive the implant, to a closed state in which the implant is enclosed within the implant receiving region 66. FIG. 6 illustrates the body 64 in a closed state. The body 64 may include a pivot 68 that portions of the body 64 are configured to pivot about to move the body between an open state and a closed state. For example, the body 64 may include a first portion 70 that may form an upper portion of the body 64 and the may include a second portion 72 that may form a lower portion of the body 64. The implant receiving region 66 accordingly may be split into two portions and opened when the portions 70, 72 are moved away from each other (rotated about the pivot 68 away from each other). The pivot 68 may extend parallel to the axis of the implant receiving region 66 such that the portions 70, 72 rotate about an axis extending parallel to the axis of the implant receiving region 66. A lock 74 may further be provided to lock the portions 70, 72 together in the closed state. The lock 74 may be releasable, and may be in the form of a latch, a clip, or other form of lock, as desired. The crimping device 62 may otherwise operate similarly as the crimping device 10 as shown in FIG. 1. For example, the crimping device 62 may include a port 76a configured similarly as the port 28 shown in FIG. 1.

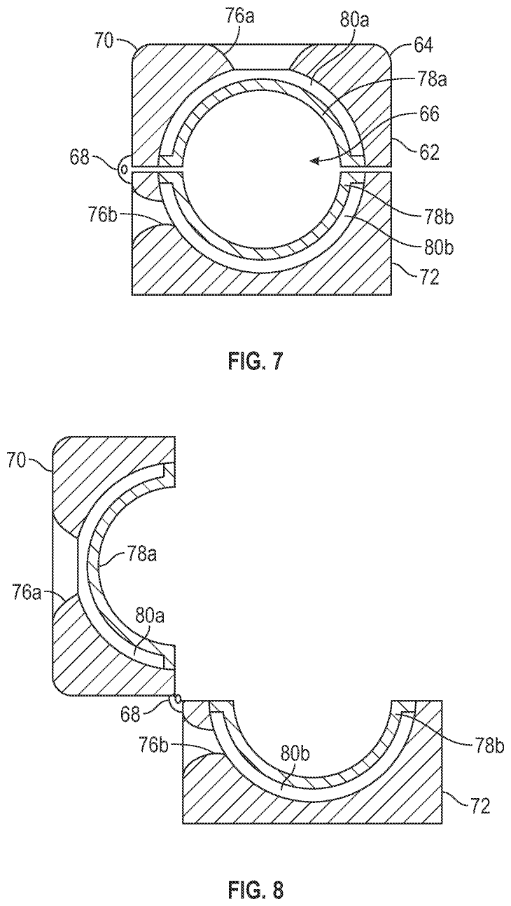

FIG. 7 illustrates a cross sectional view of the crimping device 62 along line VII-VII shown in FIG. 6. Two separate movable walls 78a, 78b may be provided that surround the implant receiving region 66. The two separate movable walls 78a, 78b form a cylindrical shape and extend around the implant receiving region 66. Two separate fluid chambers 80a, 80b may be provided that surround the respective movable walls 78a, 78b. The movable walls 78a, 78b may be separate to allow the portions 70, 72 of the body 64 to open to the open state as shown in FIG. 8. A separate port 76b may be provided that allows the fluid chamber 80b to be filled with fluid. Separate fluid conduits may couple to the ports 76a, b, yet may be coupled together (via a Y connector or another form of coupling) to provide equal fluid pressure in the fluid chambers 80a, 80b.

FIG. 7 illustrates the body 64 in the closed state, in which an implant would be enclosed within the implant receiving region 66.

FIG. 8 illustrates the body 64 in the open state in which the implant receiving region 66 is configured to receive the implant. The first portion 70 has rotated about the pivot 68 to the open state to open the implant receiving region 66 lengthwise. In such a configuration, the implant may be positioned within the implant receiving region 66 or removed from the implant receiving region 66.

In operation, the implant may be inserted into the implant receiving region 66 via an opening (for example via an opening 42 as shown in FIG. 3), or may be inserted into the implant receiving region 66 with the body 64 in the open state. The body 64 may then be moved to the closed state to enclose the implant within the implant receiving region 66. The lock 74 may be locked with the body 64 in the closed state. The fluid chambers 80a, 80b may then be filled to compress the implant with the movable walls 78a, 78b. Upon the implant being compressed to the desired amount, the implant may be slid out of the implant receiving region 66 via an opening (for example via an opening 42 as shown in FIG. 3), or the body 64 may be moved to the open state to allow the compressed implant to be removed from the implant receiving region 66. Various other configurations and methods of use of the crimping device 62 may be provided as desired.

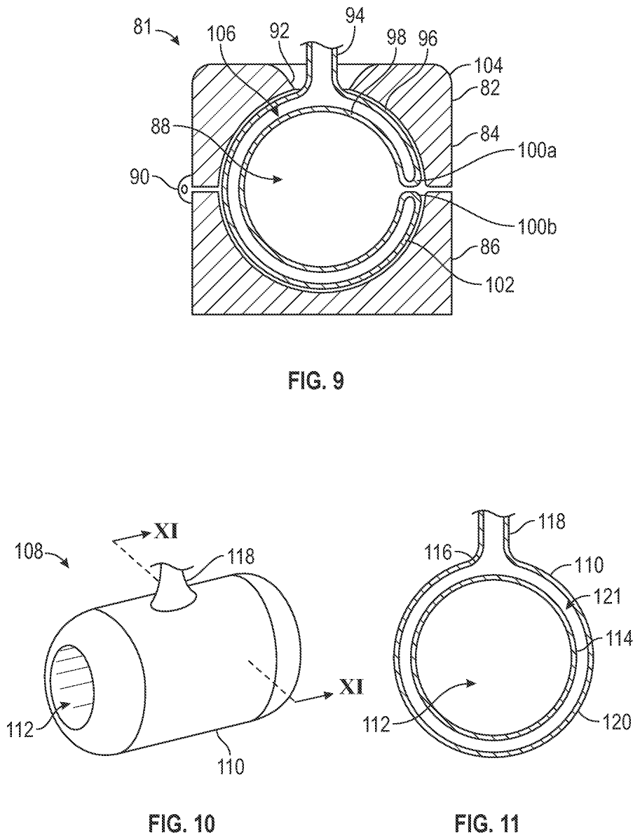

FIG. 9 illustrates a cross sectional view (for example a view as shown in FIG. 7), in which a crimping device 81 has a body 82 with two portions 84, 86 that are configured to move from an open state in which the implant receiving region 88 is configured to receive the implant, to a closed state in which the implant is enclosed within the implant receiving region 88. The portions 84, 86 may be configured to pivot about a pivot 90 in a similar manner as discussed regarding the portions of FIGS. 6-8. The body 82, however, includes a single port 92 that allows a fluid conduit 94 to couple to a bladder 96. The bladder 96 may include a movable wall 98 that operates similarly as the movable wall 30 shown in FIG. 2. The bladder 96, however, includes two ends 100a, 100b that meet, and includes a bladder wall 102 that faces towards the inner surface of the housing 104 and may press against the housing 104 as the fluid chamber 106 of the bladder 96 fills with fluid. As such, upon the portions 84, 86 moving to the open state, the bladder 96 may form a single body that allows the portions 84, 86 to open. Further, the bladder 96 may be filled via a single port 92, although in other embodiments, additional ports may be utilized as desired. The crimping device 81 may otherwise be operated in a similar manner as discussed regarding the crimping device 62.

FIGS. 10 and 11 illustrate an embodiment of a crimping device 108 having a body 110 in the form of a cuff that extends around the implant receiving region 112. The cuff may comprise a bladder that is configured to be filled with fluid in a similar manner as discussed in regard to the bladder 96 shown in FIG. 9. The cuff, however, may extend entirely around the implant receiving region 112. FIG. 11, for example, illustrates a cross sectional view of the cuff along line XI-XI in FIG. 10. The cuff may comprise a continuous bladder having a movable wall 114 that operates similarly as the movable wall 30 shown in FIG. 2. The bladder may be filled via the port 116 and fluid conduit 118. The movable wall 114 may apply the hydraulic force to compress an implant positioned within the implant receiving region 112. An outer bladder wall 120 may be non-expandable, to allow for expansion of the movable wall 114 in the direction towards the implant upon the fluid chamber 121 being filled with fluid. In other embodiments, the outer bladder wall 120 may be partially expandable or fully expandable as desired. The cuff may comprise a single body as shown in FIG. 11, or may comprise a body having separated ends as shown for example with the bladder 96 of FIG. 9. In an embodiment in which the body 110 has separated ends, the ends may be secured together upon the fluid chamber 121 being filled with fluid, for example, a lock or other device may be utilized to hold the body 110 closed. The body 110 accordingly may move between an open state and a closed state in such an embodiment.

FIG. 12 illustrates a schematic view of an embodiment in which a controller 122 is configured to operate the hydraulic actuator 38. The controller 122 may include a processor 124, a memory 126, an input device and/or output device 128, and a power source 130. The controller 122 may be configured to operate a motor 132 that may be configured to operate the hydraulic actuator 38.

The power source 130 may be configured to provide power to the controller 122, and may comprise a variety of forms of power sources, including batteries, capacitors, other forms of power cells, or an AC terminal (for example, a wall plug) for coupling to a wall socket or the like. Other forms of power sources may be utilized as desired.

The input device and/or output device 128 may be configured to receive or transmit signals with components. The input device and/or output device 128 may comprise an electrical port or terminal (for example, a USB port or other form of port), and in embodiments may comprise a wireless communication device for communicating wirelessly with components, such as a Wi-Fi or Bluetooth device or other device configured for wireless communication. The input device and/or output device 128 may be configured to transmit and receive information via the Internet or other form of communication medium. The input device and/or output device 128 may be configured to receive or transmit power signals (for example for operating the motor 132) or other electrical signals such as control signals or signals from sensors or other components.

The memory 126 may comprise a non-transitory memory that may store programs or other data for use by the processor 124. The memory 126 may comprise read-only memory (ROM), random access memory (RAM), or other forms of memory including disk memory or solid state memory. The processor 124 may be configured to retrieve data from the memory 126 or write data to the memory 126. The memory 126, for example, may store operation profiles of fluid volume, fluid flow rate, or fluid pressure, or other features, that should be executed by the motor 132 to provide a desired compression of an implant. The operation profiles in embodiments may be specific for a type of implant to be compressed by the crimping device 134. For example, the type of implant or other properties of the implant may be input into the controller 122 via the input device 128. The memory 126 may then retrieve an operation profile for that implant, which may include a profile of fluid volume, fluid flow rate, or fluid pressure, or other features. The processor 124 may then retrieve such an operation profile and may operate the motor 132 to produce such an operation for that type of implant.

The processor 124 may comprise a central processing unit (CPU) or other form of processor. The processor 124 may comprise a single processor or multiple processors as desired. In embodiments, the processor 124 may be remote from other components of the crimping device 134, for example, in a cloud computing environment or the like. The processor 124 may be configured to operate the motor 132 and accordingly control operation of hydraulic actuator 38 and the compression of the implant. The processor 124 may be configured to operate a program to operate the hydraulic actuator 38, for example, a program including an operation profile as discussed in regard to the memory 126.

The motor 132 may be utilized to operate the hydraulic actuator 38, for example, by moving the piston 48 in a desired manner. The motor may take a variety of forms, including a stepper motor, linear motor, servo motor, or other form of motor as desired.

The crimping device 134 may further utilize sensors 136, 138. The sensors 136, 138 may be configured to provide an output to a user to indicate a state of operation of the crimping device 134, and may be configured to provide feedback to the processor 124. The sensors 136, 138 may comprise one or more of a force sensor, a flow sensor, or an optical sensor, among other forms of sensors. For example, a force sensor may be utilized to detect an amount of fluid pressure provided in the fluid chamber 36. A location for such a sensor may comprise the location of sensor 138 within the fluid chamber 36, among other locations. Further, a force sensor may be positioned within the fluid chamber of the hydraulic actuator, or within a fluid conduit, among other locations. The force sensor may provide a signal to a user indicating that a certain pressure has been reached during the compression process. Such a signal may indicate to a user whether the desired fluid pressure is provided during the compression process. Further, such a signal may be provided as feedback to the processor 124, for the processor 124 to determine if a desired fluid pressure profile is being met. The processor 124 accordingly may utilize the feedback to adjust the pressure provided by the hydraulic actuator 38 by operating the motor 132 in a desired manner.

A force sensor may further comprise a sensor indicating an operation of the motor 132, to determine if a desired amount of force is provided by the motor 132. Such a signal from a sensor may be provided to a user or may be provided as feedback to the processor 124. A force sensor may be positioned within the motor 132 in such an embodiment.

The sensors 136, 138 may further comprise a flow sensor. A flow sensor may be utilized to determine an amount of fluid flow through a portion of the crimping device 134. Such a location for a flow sensor may comprise the location of the sensor 136, among other locations. The flow sensor may provide a signal to a user indicating that a certain fluid flow has been reached during the compression process. Such a signal may indicate to a user whether the desired fluid flow is provided during the compression process. Further, such a signal may be provided as feedback to the processor 124, for the processor 124 to determine if a desired fluid flow is being met. The processor 124 accordingly may utilize the feedback to adjust the flow provided by the hydraulic actuator 38 by operating the motor 132 in a desired manner.

The sensors 136, 138 may further comprise an optical sensor. An optical sensor may be utilized to visually determine a presence or size of an implant within the implant receiving region 12. Such a location for an optical sensor may comprise the location of the sensor 138, and the movable wall 30 may be optically transparent. Other locations may be utilized for an optical sensor as desired. The optical sensor may be utilized to visually determine the presence of an implant in the implant receiving region 12 for a user or the processor 124 to determine whether to initiate the compression process. For example, the optical sensor may operate as a safety device to prevent compression if no implant is present.

The optical sensor may be utilized to detect the size of the implant to determine if a certain amount of compression has been provided. For example, the optical sensor may view the implant during compression and determine if the size of the implant has been reduced to the desired compressed size. The sensor may provide a signal to a user indicating that such features have been provided. Further, such signals may be provided as feedback to the processor 124, for the processor 124 to determine if an implant is present and if a desired amount of compression has been provided. The processor 124 accordingly may utilize the feedback to control the hydraulic actuator 38 by operating the motor 132 in a desired manner. For example, if a desired amount of compression has not yet been met, then the processor 124 may continue to operate the motor 132. If a desired amount of compression has been met then the processor 124 may cease operating the motor 132.

The processor 124 may load and operate certain operation profiles from the memory 126 based on the type of implant being compressed, and may utilize the feedback from the sensors to determine whether the operation profile is being met during the compression process. The processor 124 may adjust operation of the motor 132 based on the feedback to provide a desired operation of the motor 132 and the hydraulic actuator 38 according to the operation profile.

The output from the sensors 136, 138 may also be provided to a user on an output device such as a display screen, lights, haptic feedback device, or other form of device for providing an output to a user. The user may be able to determine whether the desired operation profile is followed during the implant compression process based on the signals provided on the output device. A user may be able to adjust operation of the hydraulic actuator 38 based on the signals provided on the output device.

Power and control signal conduits 140 may couple the motor 132 and sensors 136, 138 to the controller 122 via the input device and/or output device 128.

A controller, motor, and sensors, or other features of the embodiment shown in FIG. 12, may be utilized with any embodiment of crimping device disclosed herein. For example, a controller, motor, and sensors may be utilized with an embodiment including a hydraulic actuator 39 as shown in FIGS. 21 and 22, among other embodiments disclosed herein.

The crimping devices disclosed herein, including but not limited to the crimping device 134 shown in FIG. 12, may be utilized to compress a variety of different types of implant. FIG. 13, for example, illustrates an implant 141 including couplers 142 in the form of tabs at a proximal end of the implant 141. The couplers 142 may be positioned at the ends of struts 144 of a frame 146 of the implant 141. The implant 141 may include proximal anchors 148 and may include distal anchors 150. The anchors may be configured to secure the implant to a native valve location. The implant 141 may comprise a prosthetic replacement mitral heart valve, and the distal anchors 150 may extend over leaflets of the native mitral valve. The proximal anchors 148 may be positioned on the atrial side of the native mitral valve. The implant 141 may include a skirt 152 and may extend around an axis 154.

The implant 141 may comprise a self-expanding implant, and may be configured to expand within a subject upon being released from an implant retention area of a delivery apparatus. For example, the self-expanding anchor may have a capsule covering the implant and then the capsule may be retracted from the implant to uncover the self-expanding implant and allow the implant to expand. Such an implant may be made of a nitinol material (e.g., a nitinol frame) or other shape memory material as desired. FIG. 13 illustrates the implant 141 in an expanded or deployed state.

Referring to FIG. 12, the crimping device 134 may be utilized to compress a self-expanding implant 141. The implant 141 as shown in FIG. 12 may extend over the elongate shaft of the delivery apparatus 156 and may be positioned proximate a coupler 158 configured to couple to the end couplers 142, and may be positioned proximate a capsule 160 for extending over and covering the implant 141 in a compressed or undeployed state. The implant 141 may be inserted into the implant receiving region 12 and may be compressed such that the end couplers 142 extend outward for access by the coupler 158. Upon the implant 141 being compressed, the end couplers 142 may be coupled to the coupler 158 on the elongate shaft of the delivery apparatus 156. The compressed implant 141 may then be retracted proximally with the capsule 160 advanced over the compressed implant 141 to retain the implant 141 in a compressed state. The implant accordingly may be positioned within an implant retention area of the elongate shaft of the delivery apparatus, which is within the capsule 160. The compressed implant 141 may thus be retained until a time for deployment to a desired location within the subject.

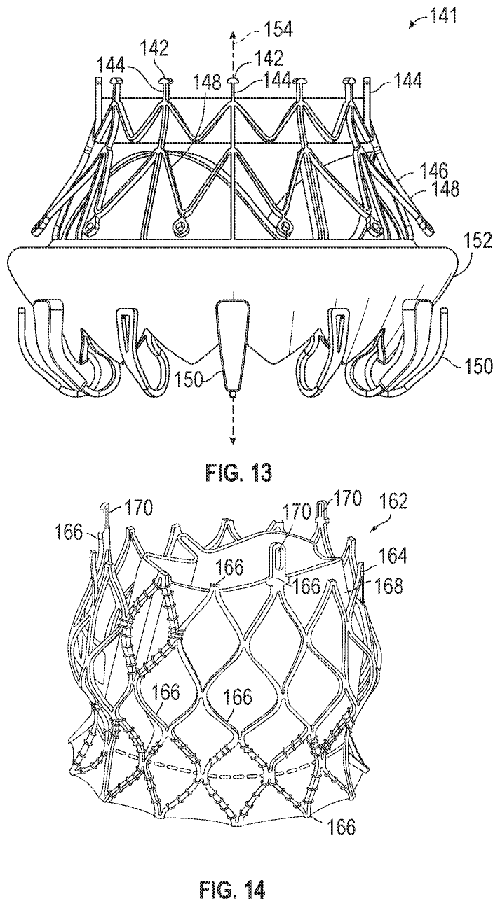

Other forms of implants may include prosthetic replacement aortic valves. FIG. 14, for example, illustrates an embodiment of a prosthetic replacement aortic valve. The implant 162 may be an expandable implant as shown in FIG. 14, which may be configured to be expanded to be placed in position within the native valve location. The implant 162 may include a frame 164 including a plurality of supports 166 configured to be compressed for positioning within the delivery apparatus and configured to be expanded at the desired time. The frame 164 may support prosthetic valve leaflets 168 that operate in lieu of the native valve leaflets. The frame 164 may include couplers 170 for coupling to the delivery apparatus, to retain the implant 162 to the delivery apparatus until deployment is desired. The couplers 170 may comprise apertures as shown in FIG. 14, or may have other forms as desired.

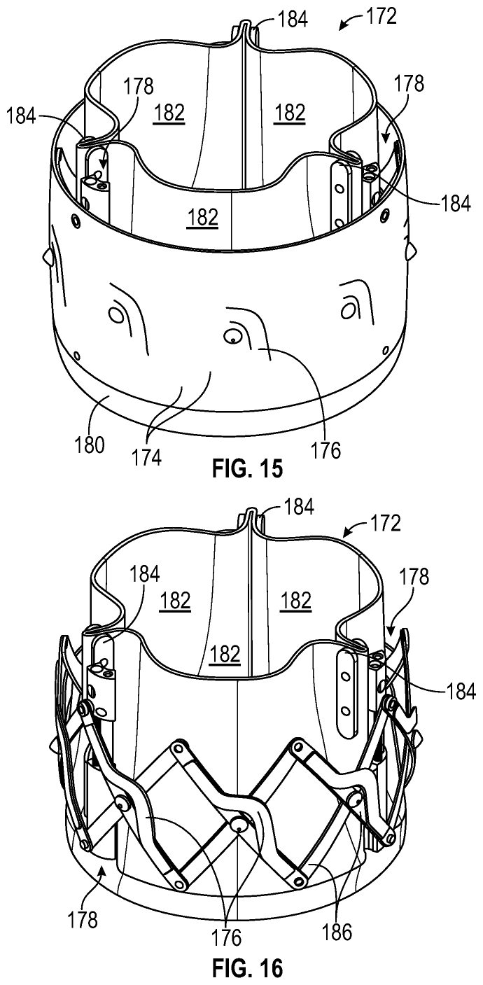

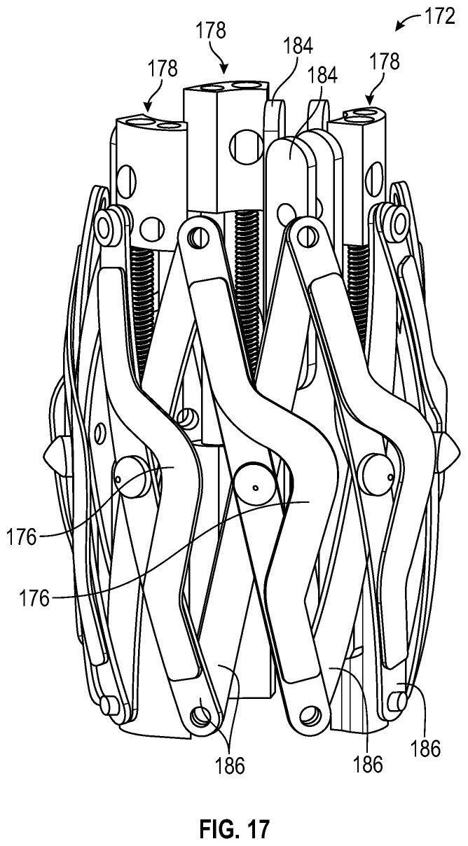

Other forms of implants may include mechanically expandable implants. A mechanically expandable implant may expand due to operation of mechanical assembly. An example of such an implant is disclosed in U.S. Pat. No. 9,913,716, filed Jan. 24, 2017 and issued Mar. 13, 2018, the entire contents of which are incorporated herein. FIGS. 72, 77, and 81 of U.S. Pat. No. 9,913,716 are reproduced here as FIGS. 15-17. The implant may include a prosthetic replacement heart valve assembly 172, a stent lattice 174, graft enclosures 176, jack assemblies 178, graft material 180, valve leaflets 182, and commissure plates 184. A cover is removed in FIG. 16 to show struts 186. FIG. 17 illustrates the implant with the cover removed, and in a compressed state. Any of the crimping devices disclosed herein may be utilized to move the implant to a compressed state as shown in FIG. 17. A mechanical assembly may then be utilized to expand the implant at a desired location within the subject.

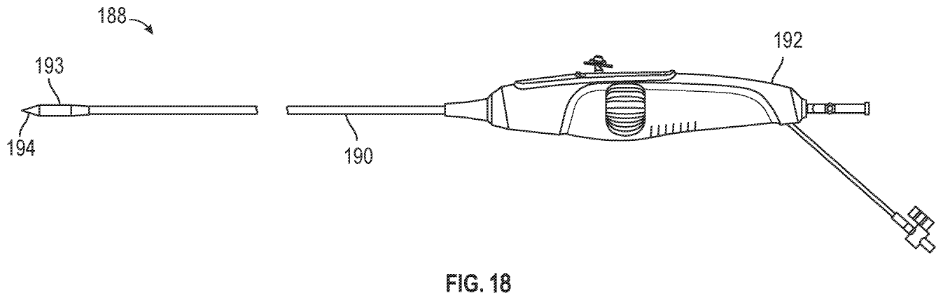

FIG. 18 illustrates a side view of a delivery apparatus 188 that may be utilized according to embodiments disclosed herein. The delivery apparatus 188 may include an elongate shaft 190 and a handle 192 positioned at a proximal end of the elongate shaft 190. A distal end of the elongate shaft 190 may include a capsule 193 covering an implant retention area. A nose cone 194 may form a tip of the elongate shaft 190. The capsule 193 may cover a compressed implant within the implant retention area.

In other embodiments, the capsule 193 may be excluded and the compressed implant may be an uncovered implant (for example a balloon expandable implant). A mechanically expandable implant may be either a covered implant (e.g., covered with a capsule) or an uncovered implant, similar to a balloon expandable implant.

The delivery apparatus shown in FIG. 18 is exemplary and other forms of delivery apparatuses may be utilized as desired.

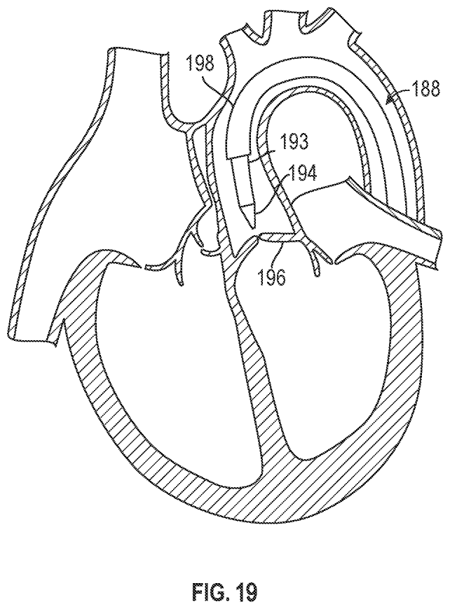

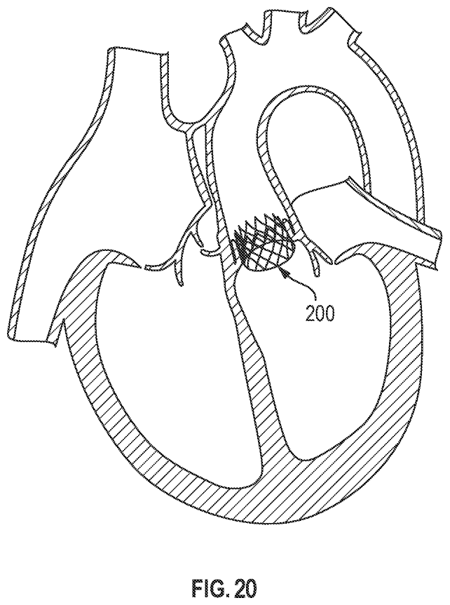

FIG. 19 illustrates an exemplary use of the delivery apparatus 188, to approach and deploy an implant to the native aortic valve 196 of a subject. The elongate shaft of the delivery apparatus 188 may be passed through the vasculature of the subject, preferably in a non-invasive manner (e.g., transcatheter percutaneous entry). A sheath 198 may further cover the elongate shaft 190 that is shown in FIG. 18. Upon reaching the desired position proximate the native aortic valve 196, the capsule 193 may be retracted to expose and deploy the implant 200 in an expanded state as shown in FIG. 20.

As discussed, various forms of implants may be utilized with the embodiments disclosed herein, including prosthetic heart valves, or other forms of implants, such as stents or filters, or diagnostic devices, among others. The implants may be expandable implants configured to move from a compressed or undeployed state to an expanded or deployed state. The implants may be compressible implants configured to be compressed inward to have a reduced outer profile and to move the implant to the compressed or undeployed state. A crimping device as disclosed herein may assist in moving the implant to the compressed or undeployed state.

The delivery apparatuses as disclosed herein may be utilized for aortic, mitral, tricuspid, and pulmonary replacement and repair as well. The delivery apparatuses may comprise delivery apparatuses for delivery of other forms of implants, such as stents or filters, or diagnostic devices, among others.

The delivery apparatuses and the systems disclosed herein may be used in transcatheter aortic valve implantation (TAVI) or replacement of other native heart valves (e.g., mitral, tricuspid, or pulmonary). The delivery apparatuses and the systems disclosed herein may be utilized for transarterial access, including transfemoral access, to a subject's heart. The delivery apparatuses and systems may be utilized in transcatheter percutaneous procedures, including transarterial procedures, which may be transfemoral or transjugular. Transapical procedures, among others, may also be utilized.

In various embodiments described herein, delivery apparatuses and methods may be deployed or performed within a subject. Subjects include (but are not limited to) medical patients, veterinary patients, animal models, cadavers, and simulators of the cardiac and vasculature system (e.g., anthropomorphic phantoms and explant tissue). Accordingly, various embodiments are directed to methods for medical procedures, practice of medical procedures, and/or training of medical procedures. Simulators may include a simulation of whole or partial vasculature system, a whole or partial heart, and/or whole or partial components of the vasculature system (e.g., whole or partial ascending aorta). References to native tissue (e.g., native heart valve) refer to preexisting structures within the subject, such as (for example) native tissue of a patient or a component of a simulator.

Features of embodiments may be modified, substituted, excluded, or combined across embodiments as desired.

The embodiments of crimping devices disclosed herein are not limited to single bodies configured to apply a hydraulic force. Rather multiple bodies (one or more bodies) may be positioned about an implant receiving region and configured to apply the hydraulic force radially upon the implant. Further, the bodies are not limited to the configurations disclosed herein. Other configurations may be utilized including a series of pistons configured to apply the hydraulic force radially upon the implant. Other configurations may be utilized as desired.

In addition, the methods herein are not limited to the methods specifically described, and may include methods of utilizing the systems and apparatuses disclosed herein. The steps of the methods may be modified, excluded, or added to, with systems, apparatuses, and methods disclosed herein.

The features of the embodiments disclosed herein may be implemented independently of the crimping devices, or independent of other components disclosed herein. The various apparatuses of the system may be implemented independently.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of systems, apparatuses, and methods as disclosed herein, which is defined solely by the claims. Accordingly, the systems, apparatuses, and methods are not limited to that precisely as shown and described.

Certain embodiments of systems, apparatuses, and methods are described herein, including the best mode known to the inventors for carrying out the same. Of course, variations on these described embodiments will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described embodiments in all possible variations thereof is encompassed by the systems, apparatuses, and methods unless otherwise indicated herein or otherwise clearly contradicted by context.

Groupings of alternative embodiments, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A crimping device for an implant, the crimping device comprising:
an implant receiving region; and
one or more bodies positioned about the implant receiving region and configured to apply a hydraulic force radially upon the implant within the implant receiving region to compress the implant.

2. The crimping device of claim 1, wherein the one or more bodies comprise a cuff extending around the implant receiving region.

3. The crimping device of claim 1, wherein the one or bodies are configured to be inflated with fluid to reduce a size of the implant receiving region and to apply the hydraulic force radially upon the implant within the implant receiving region.

4. The crimping device of claim 3, wherein the one or more bodies are configured for the fluid to be withdrawn from the one or more bodies to reduce a size of the one or more bodies and increase the size of the implant receiving region.

5. The crimping device of claim 1, wherein the one or more bodies include a fluid chamber and a movable wall positioned between the implant receiving region and the fluid chamber.

6. The crimping device of claim 1, wherein the implant receiving region has a cylindrical shape, and the one or more bodies include one or more movable walls forming a cylindrical shape and extending around the implant receiving region.

7. The crimping device of claim 1, further comprising a fluid conduit coupled to the one or more bodies and configured to transfer fluid to or from the one or more bodies.

8. The crimping device of claim 1, further comprising a hydraulic actuator configured to transfer fluid to or from the one or more bodies.

9. A system comprising:
a delivery apparatus configured to deliver an implant to a location within a subject and including an elongate shaft having an implant retention area;
an implant configured to be compressed and expanded, and configured to be retained at the implant retention area and deployed to the location within the subject; and
a crimping device having an implant receiving region and one or more bodies configured to apply a hydraulic force radially upon the implant within the implant receiving region to compress the implant.

10. The system of claim 9, wherein the implant receiving region is configured to receive the elongate shaft of the delivery apparatus, and the one or more bodies are configured to compress the implant to the elongate shaft.

11. The system of claim 10, wherein the elongate shaft includes a capsule configured to extend over the implant.

12. The system of claim 9, wherein the one or more bodies are expandable and are configured to be filled with fluid to apply the hydraulic force radially upon the implant.

13. The system of claim 12, wherein the one or more bodies are configured for the fluid to be withdrawn from the one or more bodies.

14. The system of claim 13, wherein the implant receiving region has a cylindrical shape, and the one or more bodies include one or more movable walls forming a cylindrical shape and extending around the implant receiving region.

15. The system of claim 9, wherein the implant comprises one or more of a mechanically expandable implant, a self-expanding implant, or a balloon expandable implant.

16. The system of claim 9, further comprising a hydraulic actuator configured to transfer fluid to or from the one or more bodies.

17. The system of claim 16, wherein the hydraulic actuator includes a fluid chamber and a piston configured to move within the fluid chamber.

18. The system of claim 17, further comprising a controller configured to operate the hydraulic actuator, and the controller includes a processor configured to operate a program to operate the hydraulic actuator, and the system further comprises one or more sensors configured to provide feedback to the processor, the one or more sensors include one or more of a force sensor, a flow sensor, or an optical sensor.

19. A method comprising:
positioning an implant within an implant receiving region of a crimping device, the crimping device including one or more bodies configured to apply a hydraulic force to the implant; and
applying the hydraulic force radially upon the implant with the one or more bodies to compress the implant within the implant receiving region.

20. The method of claim 19, further comprising moving the one or more bodies from an open state in which the implant receiving region is configured to receive the implant to a closed state in which the implant is enclosed within the implant receiving region.
